SEQUENCE LISTING (25 sequences)

SEQ ID NO: 1 - protein, 16 aa, Artificial Sequence
  source: /note=“Description of Artificial Sequence Synthetic peptide”
DTYYYDSGDYEDAFDI

SEQ ID NO: 2 - protein, 16 aa, Artificial Sequence
  source: /note=“Description of Artificial Sequence Synthetic peptide”
YIHYSRSTNSNPALKS

SEQ ID NO: 3 - protein, 5 aa, Artificial Sequence
  source: /note=“Description of Artificial Sequence Synthetic peptide”
GYYWS

SEQ ID NO: 4 - protein, 9 aa, Artificial Sequence
  source: /note=“Description of Artificial Sequence Synthetic peptide”
QTWGAGIRV

SEQ ID NO: 5 - protein, 11 aa, Artificial Sequence
  source: /note=“Description of Artificial Sequence Synthetic peptide”
VNRDGSHIRGD

SEQ ID NO: 6 - protein, 12 aa, Artificial Sequence
  source: /note=“Description of Artificial Sequence Synthetic peptide”
TLSSGHSNYAIA

SEQ ID NO: 7 - protein, 124 aa, Artificial Sequence
  source: /note=“Description of Artificial Sequence Synthetic polypeptide”
QVQLQESGPGLVKPSETLSLTCTVSGGSISGYYWSWIRQPPGKGLEWIGYIHYSRSTNSNPALKSRVTISSDTSKNQLSLRLSSVTAADTAVYYCARDTYYYDSGDYEDAFDIWGQGTMVTVSS

SEQ ID NO: 8 - protein, 112 aa, Artificial Sequence
  source: /note=“Description of Artificial Sequence Synthetic polypeptide”
QLVLTQSPSASASLGASVKLTCTLSSGHSNYAIAWHQQQPGKGPRYLMKVNRDGSHIRGDGIPDRFSGSTSGAERYLTISSLQSEDEADYYCQTWGAGIRVFGGGTKLTVLG

SEQ ID NO: 9 - protein, 16 aa, Artificial Sequence
  source: /note=“Description of Artificial Sequence Synthetic peptide”
DTYYESSGHWFDGLDV

SEQ ID NO: 10 - protein, 16 aa, Artificial Sequence
  source: /note=“Description of Artificial Sequence Synthetic peptide”
YIHYSGSTNSNPSLKS

SEQ ID NO: 11 - protein, 5 aa, Artificial Sequence
  source: /note=“Description of Artificial Sequence Synthetic peptide”
NYYWS

SEQ ID NO: 12 - protein, 9 aa, Artificial Sequence
  source: /note=“Description of Artificial Sequence Synthetic peptide”
QTWGPGIRV

SEQ ID NO: 13 - protein, 11 aa, Artificial Sequence
  source: /note=“Description of Artificial Sequence Synthetic peptide”
VKSDGSHSKGD

SEQ ID NO: 14 - protein, 12 aa, Artificial Sequence
  source: /note=“Description of Artificial Sequence Synthetic peptide”
TLDSEHSRYTIA

SEQ ID NO: 15 - protein, 124 aa, Artificial Sequence
  source: /note=“Description of Artificial Sequence Synthetic polypeptide”
QVQLQESGPGLVKPSETLSLTCTVSGGSISNYYWSWIRQSPGRGLEWIGYIHYSGSTNSNPSLKSRVTISVDTSKNQVSLKLGSVTAADTAIYYCARDTYYESSGHWFDGLDVWGQGTSVTVSS

SEQ ID NO: 16 - protein, 111 aa, Artificial Sequence
  source: /note=“Description of Artificial Sequence Synthetic polypeptide”
QPVLTQSPSASASLGASVKLTCTLDSEHSRYTIAWHQQQPEKGPRYLMKVKSDGSHSKGDGITDRFSGSSSGAERYLTISSLQSEDEADYYCQTWGPGIRVFGGGTKLTVL

SEQ ID NO: 17 - protein, 15 aa, Artificial Sequence
  source: /note=“Description of Artificial Sequence Synthetic peptide”
DYYETSGYAYDDFAI

SEQ ID NO: 18 - protein, 17 aa, Artificial Sequence
  source: /note=“Description of Artificial Sequence Synthetic peptide”
WVSTYNGRTNYAQKFRG

SEQ ID NO: 19 - protein, 5 aa, Artificial Sequence
  source: /note=“Description of Artificial Sequence Synthetic peptide”
NFGIS

SEQ ID NO: 20 - protein, 9 aa, Artificial Sequence
  source: /note=“Description of Artificial Sequence Synthetic peptide”
QTWGPGIRV

SEQ ID NO: 21 - protein, 11 aa, Artificial Sequence
  source: /note=“Description of Artificial Sequence Synthetic peptide”
VNSDGSHTKGD

SEQ ID NO: 22 - protein, 12 aa, Artificial Sequence
  source: /note=“Description of Artificial Sequence Synthetic peptide”
TLSSGHSTYAIA

SEQ ID NO: 23 - protein, 124 aa, Artificial Sequence
  source: /note=“Description of Artificial Sequence Synthetic polypeptide”
QVQLVQSGAEMKRPGASVKVSCKASGYTFTNFGISWVRQAPGQGLEWIGWVSTYNGRTNYAQKFRGRVTMTTDTSTNTAYMELRSLGSDDTAVFYCARDYYETSGYAYDDFAIWGQGTLVTVSS

SEQ ID NO: 24 - protein, 112 aa, Artificial Sequence
  source: /note=“Description of Artificial Sequence Synthetic polypeptide”
QLVLTQSPSASASLGASVKLTCTLSSGHSTYAIAWHQQQPLRGPRFLMKVNSDGSHTKGDGIPDRFSGSSSGAERYLSISSLQSEDESDYYCQTWGPGIRVFGGGTKLTVLG

SEQ ID NO: 25 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2015/0086601 A1
(43) Pub. Date: Mar. 26, 2015

(54) METHODS FOR PREVENTING BIOFILM FORMATION

(71) Applicant: SANOFI, Paris (FR)

(72) Inventors: Astrid Rey, Paris (FR); Sylvie Lefort, Paris (FR); Laurent Fraisse, Paris (FR); Jeremy Yethon, Boston, MA (US)

(73) Assignee: SANOFI, Paris (FR)

(21) Appl. No.: 14/396,897

(22) PCT Filed: May 6, 2013
(86) PCT No.: PCT/US2013/039724

§ 371 Date: Oct. 24, 2014

Related U.S. Application Data
(60) Provisional application No. 61/643,650, filed on May 7, 2012.

(30) Foreign Application Priority Data
Mar. 18, 2013 (FR) 1352399

(51) Int. Cl. C07K 16/12; A61L 31/16; A61L 29/16

(52) U.S. Cl. CPC C07K 16/1271; A61L 29/16; A61L 31/16; C07K 2317/21; C07K 2317/565; A61L 2300/256
USPC 424/423; 424/165.1

(57) ABSTRACT

Provided are methods for the treatment or prevention of microbial infections (e.g., nocosomial infection) in which the underlying pathology involves a PNAG-containing microbial bio film. The methods the invention generally involve administering to the subject an effective amount of an antibody that specifically binds to PNAG and disrupt or inhibit formation of PNAG-containing microbial bio films. Such methods are particularly useful for the treatment of nosocomial staphylococcus (e.g., S. epidermidis and S. aureus) infections.

### RELATED APPLICATION

This application claims priority to U.S. Provisional Application No. 61/643,650, filed May 7, 2012. The content of this application is hereby incorporated by reference in its entirety.

### BACKGROUND OF THE INVENTION

A biofilm is an aggregate of microorganisms in which the individual cells adhere to each other on a surface, usually embedded within a self-produced polymeric matrix. Biofilms have been found to be involved in a wide variety of microbial infections in the human body, including common conditions such as urinary tract infections, catheter infections, middle-ear infections, formation of dental plaque, gingivitis, as well as less common but more lethal conditions such as endocarditis, lung infections in cystic fibrosis, and infections of permanent indwelling devices, such as joint prostheses, heart valves and intrauterine devices. It has also been noted that bacterial biofilms may impair cutaneous wound healing and reduce topical antibacterial efficiency in healing or treating infected skin wounds.

In many bacteria, including staphylococci, the main molecule responsible for intercellular adhesion is the glucosamine polymer, poly-N-acetylglucosamine (PNAG). PNAG is a beta-1-6-linked N-acetylglucosamine polymer, which together with other polymers such as teichoic acids and proteins forms the major part of the extracellular matrix of the bacterial biofilm.

Staphylococcus epidermidis and S. aureus are the most frequent causes of nosocomial infections (e.g., on indwelling medical devices), which characteristically involve biofilms. Biofilm formation by S. epidermidis and S. aureus is particularly problematic in that it protects the bacteria from attack by antibiotics and the host immune system, thereby rendering the infection difficult to treat.

Accordingly, there is a need in the art for alternative methods of treating or preventing S. epidermidis or S. aureus infections. Specifically, there is a need for methods that act by disrupting or inhibiting biofilm formation by these bacteria.

### SUMMARY OF THE INVENTION

The present invention provides methods for the treatment or prevention of microbial infections (e.g., nocosomial infection) in which the underlying pathology involves a PNAG-containing microbial biofilm. The methods of the invention generally involve administering to the subject an effective amount of an antibody that specifically binds to PNAG and disrupts or inhibits formation of PNAG-containing microbial biofilms. Such methods are particularly useful for the treatment of nosocomial staphylococcus (e.g., S. epidermidis and S. aureus) infections.

Accordingly, in one aspect, the invention provides a method of preventing a nocosomial infection comprising, the method generally comprising: identifying a subject (e.g., human subject) at risk of developing an poly-N-acetyl glucosamine (PNAG)-containing microbial biofilm from a medical procedure; and administering to the subject an effective amount of an antibody that specifically binds to PNAG and inhibits PNAG-containing microbial biofilm formation.

In certain embodiments, the nocosomial infection is a lung infection, joint infection, endocardial infection, skin infection, soft tissue infection, or septicemia.

In certain embodiments, the antibody is administered between about 0 and 240 hours prior to the medical procedure.

In certain embodiments, the medical procedure is the installation of a surgical implant in the subject, e.g., a stent, catheter, cannula, prosthesis, or pace-maker.

In certain embodiments, the antibody is administered systemically. In other embodiments, the antibody is administered locally to the site of implantation of the surgical implant.

In certain embodiments, the antibody is coated on the surgical implant or adhered to the surgical implant.

In certain embodiments, the antibody is administered in a controlled release formulation.

In certain embodiments, the biofilm comprises Staphylococcus, e.g., S. epidermidis and S. aureus.

In certain embodiments, the antibody is a human antibody.

In certain embodiments, the antibody comprises a VH domain comprising the HCDR1, HCDR2, and HCDR3 amino acid sequences set forth in SEQ ID Nos: 1, 2, and 3, respectively.

In certain embodiments, the antibody comprises a VL domain comprising the LCDR1, LCDR2, and LCDR3 amino acid sequences set forth in SEQ ID Nos: 4, 5, and 6, respectively.

In certain embodiments, the antibody comprises the VH domain sequence set forth in SEQ ID No: 7.

The method of any one of the preceding claims, wherein the antibody comprises the VL domain sequence set forth in SEQ ID No: 8.

In another aspect, the invention provides a method of inhibiting formation of a poly N-acetyl glucosamine (PNAG)-containing microbial biofilm on a surgical implant in a subject (e.g., a human subject), the method generally comprising administering to the subject an effective amount of an antibody that specifically binds to PNAG prior to implantation of the surgical implant into the subject.

In certain embodiments, the surgical implant is a stent, catheter, cannula, prosthesis, or pace-maker.

In certain embodiments, the antibody is administered between about 0 and 240 hours prior to the medical procedure.

In certain embodiments, the antibody is administered systemically. In other embodiments, the antibody is administered locally to the site of implantation of the surgical implant.

In certain embodiments, the antibody is coated on the surgical implant or adhered to the surgical implant.

In certain embodiments, the antibody is administered in a controlled release formulation.

In certain embodiments, the biofilm comprises Staphylococcus, e.g., S. epidermidis and S. aureus.

In certain embodiments, the antibody is a human antibody.

In certain embodiments, the antibody comprises a VH domain comprising the HCDR1, HCDR2, and HCDR3 amino acid sequences set forth in SEQ ID Nos: 1, 2, and 3, respectively.

In certain embodiments, the antibody comprises a VL domain comprising the LCDR1, LCDR2, and LCDR3 amino acid sequences set forth in SEQ ID Nos: 4, 5, and 6, respectively.

In certain embodiments, the antibody comprises the VH domain sequence set forth in SEQ ID No: 7.

The method of any one of the preceding claims, wherein the antibody comprises the VL domain sequence set forth in SEQ ID No: 8.

In a further aspect, the invention provides a method of inhibiting formation of PNAG-containing microbial biofilm on a substrate comprising contacting the substrate with an effective amount of an antibody that specifically binds to PNAG.

In certain embodiments, the biofilm comprises Staphylococcus, e.g., S. epidermidis and S. aureus.

In certain embodiments, the antibody is a human antibody.

In certain embodiments, the antibody comprises a VH domain comprising the HCDR1, HCDR2, and HCDR3 amino acid sequences set forth in SEQ ID Nos: 1, 2, and 3, respectively.

In certain embodiments, the antibody comprises a VL domain comprising the LCDR1, LCDR2, and LCDR3 amino acid sequences set forth in SEQ ID Nos: 4, 5, and 6, respectively.

In certain embodiments, the antibody comprises the VH domain sequence set forth in SEQ ID No: 7.

The method of any one of the preceding claims, wherein the antibody comprises the VL domain sequence set forth in SEQ ID No: 8.

### BRIEF DESCRIPTION OF THE DRAWING

FIG. 1 depicts the results of in vitro assays measuring biofilm formation of S. aureus (ATCC 33592) in the presence and absence of F598.

FIG. 2 depicts the results of in vitro assays measuring biofilm formation of S. epidermidis 1457 in the presence and absence of F598.

FIG. 3 depicts the results of in vitro assays measuring biofilm formation of S. epidermidis RP62A in the presence and absence of F598.

### DETAILED DESCRIPTION

The present invention provides methods for the treatment or prevention of microbial infections (e.g., nocosomial infection) in which the underlying pathology involves a PNAG-containing microbial biofilm. The methods the invention generally involve administering to the subject an effective amount of an antibody that specifically binds to PNAG and disrupt or inhibit formation of PNAG-containing microbial biofilms. Such methods are particularly useful for the treatment of nosocomial staphylococcus (e.g., S. epidermidis and S. aureus) infections.

### I. DEFINITIONS

In order that the present invention may be more readily understood, certain terms are first defined.

As used herein, the terms “poly-N-acetyl glucosamine” or “PNAG” refer to a polymer of N-acetyl glucosamine monomers linked via a beta 1-6 linkage. The terms also encompass partially or fully deacylated poly-N-acetyl glucosamine.

As used herein, the term “PNAG-containing microbial biofilm” refers to sessile aggregate of microorganisms embedded in a PNAG matrix.

As used herein, the term “nosocomial infection” refers to infection acquired in a hospital or from a medical procedure performed inside or outside of a hospital. Exemplary nocosomial infections include sepsis (bloodstream infection), surgical site infection, or hospital-acquired pneuomia.

As used herein, the term “preventing a nocosomial infection” refers to an inhibition or reduction in the severity of an infection.

As used herein, the term “antibody” refers to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated V_{H}) and a heavy chain constant region. The heavy chain constant region comprises three domains, C_{H}1, C_{H}2 and C_{H}3. Each light chain comprises a light chain variable region (abbreviated V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4.

As used herein, the term “antigen-binding portion” of an antibody include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Non-limiting examples of antigen-binding portions include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR)). Other engineered molecules, such as diabodies, triabodies, tetrabodies and minibodies, are also encompassed within the expression “antigen-binding portion.”

As used herein, the term “CDR” or “complementarity determining region” means the noncontiguous antigen combining sites found within the variable region of both heavy and light chain polypeptides. These particular regions have been described by Kabat et al., J. Biol. Chem. 252, 6609-6616 (1977) and Kabat et al., Sequences of protein of immunological interest. (1991), and by Chothia et al., J. Mol. Biol. 196:901-917 (1987) and by MacCallum et al., J. Mol. Biol. 262:732-745 (1996) where the definitions include overlapping or subsets of amino acid residues when compared against each other. The amino acid residues which encompass the CDRs as defined by each of the above cited references are set forth for comparison. Preferably, the term “CDR” is a CDR as defined by Kabat, based on sequence comparisons.

As used herein the term “framework (FR) amino acid residues” refers to those amino acids in the framework region of an Ig chain. The term “framework region” or “FR region” as used herein, includes the amino acid residues that are part of the variable region, but are not part of the CDRs (e.g., using the Kabat definition of CDRs). Therefore, a variable region framework is between about 100-120 amino acids in length but includes only those amino acids outside of the CDRs.

As used herein, the term “specifically binds to” refers to the ability of an antibody or an antigen-binding fragment thereof to bind to an antigen with an Kd of at least about 1×10⁻⁶ M, 1×10⁻⁷M, 1×10⁻⁸M, 1×10⁻⁹M, 1×10⁻¹⁰ M, 1×10⁻¹¹ M, 1×10⁻¹² M, or more, and/or bind to an antigen with an affinity that is at least two-fold greater than its affinity for a nonspecific antigen.

As used herein, the term “antigen” refers to the binding site or epitope recognized by an antibody or antigen binding portion thereof.

As used herein, the term “effective amount” refers to that amount of an antibody or an antigen binding portion thereof that binds PNAG, which is sufficient to effect treatment, prognosis or diagnosis of a PNAG-containing microbial biofilm, as described herein, when administered to a subject. A therapeutically effective amount will vary depending upon the subject and disease condition being treated, the weight and age of the subject, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 ug to about 3,500 mg, about 5 ug to about 3,000 mg, about 10 ug to about 2,600 mg, about 20 ug to about 2,575 mg, about 30 ug to about 2,550 mg, about 40 ug to about 2,500 mg, about 50 ug to about 2,475 mg, about 100 ug to about 2,450 mg, about 200 ug to about 2,425 mg, about 300 ug to about 2,000 mg, about 400 ug to about 1,175 mg, about 500 ug to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of an antibody or antigen binding portion thereof, according to the invention. Dosage regiments may be adjusted to provide the optimum therapeutic response. An effective amount is also one in which any toxic or detrimental effects (i.e., side effects) of an antibody or antigen binding portion thereof are minimized and/or outweighed by the beneficial effects.

As used herein, the term “subject” includes any human or non-human animal.

### II. BIOFILM

The methods of the invention can be used to disrupt or inhibit the formation of poly N-acetyl glucosamine (PNAG)-containing microbial biofilms. PNAG-containing biofilm can be produced by a variety of microbes, including bacteria and fungi. In general, any PNAG-containing microbial biofilm can be disrupted or inhibited using the methods of the invention.

In the case of bacteria, PNAG-containing biofilms are commonly formed by, for example, Staphylococci (e.g., S. epidermis, S. aureus (e.g., Multi Drug Resistant S. aureus), S. carnosus, and S. haemolyticus). Known clinical isolates of Staphylococcus that form PNAG-containing biofilms include, without limitation, S. epidermis RP62A (ATCC number 35984), S. epidermis RP12 (ATCC number 35983), S. epidermis M187, S. carnosus TM300 (pCN27), S. aureus RN4220 (pCN27), and S. aureus MN8 mucoid.

PNAG-containing biofilms are also formed by other bacteria including but not limited to Pseudomonas aeruginosa, E. coli (e.g., E. coli O157:H7 and E. coli CFT073), Yersinia pestis, Yersinia entercolitica, Xanthomonas axonopodis, Pseudomonas fluorescens, Actinobacillus actinomycetemcomitans, Actinobacillus pleuropneumoniae, Ralstonia solanacearum, Bordetella pertussis, Bordetella parapertussis and Bordetella bronchiseptica.

### III. NOSOCOMIAL INFECTIONS

The invention provides methods for treating or preventing nosocomial infections by identifying a subject at risk of developing a poly-N-acetyl glucosamine (PNAG)-containing microbial biofilm from a medical procedure and administering to the subject an effective amount of an antibody that specifically binds to PNAG and inhibits PNAG-containing microbial biofilm formation.

Any medical procedure, whether performed inside or outside of a hospital, that confers a risk to a patient of developing a PNAG-containing microbial biofilm can be treated or prevented using the methods of the invention. Examples of such medical procedures include, without limitation, surgery and implantation of a surgical device (e.g., catheter, cannula, prosthesis, respirator, replacement heart valve, and pace-maker). Exemplary surgical devices include, without limitation, central venous catheters; peritoneal dialysis catheters; orthopedic prostheses; orthopedic mesh; intracardiac devices such as artificial valves, pacemakers, and stents; cochlear implants; breast implants; endotracheal tubes; voice prostheses; intraocular lens

The skilled artisan will appreciate that in the case of an immunosuppressed subject, the mere fact that the subject is in a hospital (or another environment that can harbor bacteria capable of PNAG-containing microbial biofilm) identifies this subject as being at risk of developing a PNAG-containing microbial biofilm (e.g., in the lung, urinary tract or in an open wound) even if they do not receive a surgical procedure.

### IV. ANTI-PNAG ANTIBODIES

Any antibody that binds to PNAG and inhibits formation of a PNAG-containing bacterial biofilm can be used in the methods of the invention. Exemplary antibody VH, VL and CDR amino acid sequences suitable for use in the invention are set forth in Table 1.

TABLE 1
VH, VL and CDR amino acid sequences
of exemplary anti-PNAG antibodies.

| | | SEQ |
|---|---|---|
| Antibody | Sequence | ID NO. |
| F598 HCDR3 | DTYYYDSGDYEDAFDI | 1 |
| F598 HCDR2 | YIHYSRSTNSNPALKS | 2 |
| F598 HCDR1 | GYYWS | 3 |
| F598 LCDR3 | QTWGAGIRV | 4 |
| F598 LCDR2 | VNRDGSHIRGD | 5 |
| F598 LCDR1 | TLSSGHSNYAIA | 6 |
| F598 VH | QVQLQESGPGLVKPSETLSLTCTVSGGSIS | 7 |
| | GYYWSWIRQPPGKGLEWIGYIHYSRSTNSN | |
| | PALKSRVTISSDTSKNQLSLRLSSVTAADT | |
| | AVYYCARDTYYYDSGDYEDAFDIWGQGTMV | |
| | TVSS | |
| F598 VL | QLVLTQSPSASASLGASVKLTCTLSSGHSN | 8 |
| | YAIAWHQQQPGKGPRYLMKVNRDGSHIRGD | |
| | GIPDRFSGSTSGAERYLTISSLQSEDEADY | |
| | YCQTWGAGIRVFGGGTKLTVLG | |
| F628 HCDR3 | DTYYESSGHWFDGLDV | 9 |
| F628 HCDR2 | YIHYSGSTNSNPSLKS | 10 |
| F628HCDR1 | NYYWS | 11 |
| F628LCDR3 | QTWGPGIRV | 12 |
| F628 LCDR2 | VKSDGSHSKGD | 13 |
| F628 LCDR1 | TLDSEHSRYTIA | 14 |
| F628VH | QVQLQESGPGLVKPSETLSLTCTVSGGSIS | 15 |
| | NYYWSWIRQSPGRGLEWIGYIHYSGSTNSN | |
| | PSLKSRVTISVDTSKNQVSLKLGSVTAADT | |
| | AIYYCARDTYYESSGHWFDGLDVWGQGTSV | |
| | TVSS | |
| F628 VL | QPVLTQSPSASASLGASVKLTCTLDSEHSR | 16 |
| | YTIAWHQQQPEKGPRYLMKVKSDGSHSKGD | |
| | GITDRFSGSSSGAERYLTISSLQSEDEADY | |
| | YCQTWGPGIRVFGGGTKLTVL | |
| F630 HCDR3 | DYYETSGYAYDDFAI | 17 |
| F630 HCDR2 | WVSTYNGRTNYAQKFRG | 18 |
| F630 HCDR1 | NFGIS | 19 |
| F630 LCDR3 | QTWGPGIRV | 20 |
| F630 LCDR2 | VNSDGSHTKGD | 21 |
| F630LCDR1 | TLSSGHSTYAIA | 22 |
| F630 VH | QVQLVQSGAEMKRPGASVKVSCKASGYTFT | 23 |
| | NFGISWVRQAPGQGLEWIGWVSTYNGRTNY | |
| | AQKFRGRVTMTTDTSTNTAYMELRSLGSDD | |
| | TAVFYCARDYYETSGYAYDDFAIWGQGTLV | |
| | TVSS | |
| F630 VL | QLVLTQSPSASASLGASVKLTCTLSSGHST | 24 |
| | YAIAWHQQQPLRGPRFLMKVNSDGSHTKGD | |
| | GIPDRFSGSSSGAERYLSISSLQSEDESDY | |
| | YCQTWGPGIRVFGGGTKLTVLG | |

In certain embodiments, the antibody, or antigen binding fragment thereof, comprises one or more CDR region amino acid sequence selected from the group consisting of SEQ ID NO: 1, 2, 3, 4, 5, 6, 9, 10, 11, 12, 13, 14, 17, 18, 19, 20, 21, and 22.

In other embodiments, the antibody, or antigen binding fragment thereof, comprises HCDR3, HCDR2 and HCDR1 region amino acid sequences selected from the group consisting of:

a) SEQ ID NO: 1, 2 and 3;

b) SEQ ID NO: 9, 10 and 11; and

c) SEQ ID NO: 17, 18 and 19, respectively.

In other embodiments, the antibody, or antigen binding fragment thereof, comprises the LCDR3, LCDR2 and LCDR1 region amino acid sequences selected from the group consisting of:

a) SEQ ID NO: 4, 5 and 6;

b) SEQ ID NO: 12, 13 and 14; and

c) SEQ ID NO: 20, 21 and 22, respectively.

In other embodiments, the antibody, or antigen binding fragment thereof, comprises the HCDR3, HCDR2, HCDR1, LCDR3, LCDR2 and LCDR1 region amino acid sequences selected from the group consisting of:

a) SEQ ID NO: 1, 2, 3, 4, 5 and 6;

b) SEQ ID NO: 9, 10, 21, 12, 13 and 14; and

c) SEQ ID NO: 17, 18, 21, 20, 21 and 22, respectively

In other embodiments, the antibody, or antigen binding fragment thereof, comprises the VH region amino acid sequences set forth in SEQ ID NO: 7, 15 and/or 23.

In other embodiments, the antibody, or antigen binding fragment thereof, comprises the VL region amino acid sequences set forth in SEQ ID NO: 8, 16, and/or 24.

In other embodiments, the antibody, or antigen binding fragment thereof, comprises the VH and VL region amino acid sequences selected from the group consisting of: SEQ ID NO: 7 and 8; SEQ ID NO: 15 and 16; and SEQ ID NO: 23 and 24, respectively.

### V. MODIFIED ANTI-PNAG ANTIBODIES

Anti-PNAG antibodies may comprise one or more modifications. Such modified forms of anti-PNAG antibodies can be made using any techniques known in the art.

### i) Reducing Immunogenicity

In some embodiments, de-immunization can be used to decrease the immunogenicity of and antibody, or antigen binding portion thereof. As used herein, the term “de-immunization” includes alteration of an antibody, or antigen binding portion thereof, to modify T cell epitopes (see, e.g., WO9852976A1, WO0034317A2). For example, VH and VL sequences from the starting antibody may be analyzed and a human T cell epitope “map” may be generated from each V region showing the location of epitopes in relation to complementarity-determining regions (CDRs) and other key residues within the sequence. Individual T cell epitopes from the T cell epitope map are analyzed in order to identify alternative amino acid substitutions with a low risk of altering activity of the final antibody. A range of alternative VH and VL sequences are designed comprising combinations of amino acid substitutions and these sequences are subsequently incorporated into a range of anti-PNAG antibodies or fragments thereof for use in the methods disclosed herein, which are then tested for function. Complete heavy and light chain genes comprising modified V and human C regions are then cloned into expression vectors and the subsequent plasmids introduced into cell lines for the production of whole antibody. The antibodies are then compared in appropriate biochemical and biological assays, and the optimal variant is identified.

### ii) Effector Functions and Fc Modifications

Anti-PNAG antibodies may comprise an antibody constant region (e.g. an IgG constant region e.g., a human IgG constant region, e.g., a human IgG1 or IgG4 constant region) which mediates one or more effector functions. For example, binding of the C1 component of complement to an antibody constant region may activate the complement system. Activation of complement is important in the opsonisation and lysis of cell pathogens. The activation of complement also stimulates the inflammatory response and may also be involved in autoimmune hypersensitivity. Further, antibodies bind to receptors on various cells via the Fc region, with a Fc receptor binding site on the antibody Fc region binding to a Fc receptor (FcR) on a cell. There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. In preferred embodiments, the anti-PNAG antibodies, or fragments thereof, bind to an Fc.gamma. receptor. In alternative embodiments, anti-PNAG antibodies may comprise a constant region which is devoid of one or more effector functions (e.g., ADCC activity) and/or is unable to bind Fcγ receptor.

Certain embodiments of the invention include anti-PNAG antibodies in which at least one amino acid in one or more of the constant region domains has been deleted or otherwise altered so as to provide desired biochemical characteristics such as reduced or enhanced effector functions, the ability to non-covalently dimerize, increased ability to localize at the site of a tumor, reduced serum half-life, or increased serum half-life when compared with a whole, unaltered antibody of approximately the same immunogenicity. For example, certain antibodies, or fragments thereof, for use in the diagnostic and treatment methods described herein are domain deleted antibodies which comprise a polypeptide chain similar to an immunoglobulin heavy chain, but which lack at least a portion of one or more heavy chain domains. For instance, in certain antibodies, one entire domain of the constant region of the modified antibody will be deleted, for example, all or part of the CH2 domain will be deleted.

In certain other embodiments, anti-PNAG antibodies comprise constant regions derived from different antibody isotypes (e.g., constant regions from two or more of a human IgG1, IgG2, IgG3, or IgG4). In other embodiments, anti-PNAG antibodies comprises a chimeric hinge (i.e., a hinge comprising hinge portions derived from hinge domains of different antibody isotypes, e.g., an upper hinge domain from an IgG4 molecule and an IgG1 middle hinge domain). In one embodiment, an anti-PNAG antibodies comprises an Fc region or portion thereof from a human IgG4 molecule and a Ser228Pro mutation (EU numbering) in the core hinge region of the molecule.

In certain anti-PNAG antibodies, the Fc portion may be mutated to increase or decrease effector function using techniques known in the art. For example, the deletion or inactivation (through point mutations or other means) of a constant region domain may reduce Fc receptor binding of the circulating modified antibody thereby increasing tumor localization. In other cases it may be that constant region modifications consistent with the instant invention moderate complement binding and thus reduce the serum half life and nonspecific association of a conjugated cytotoxin. Yet other modifications of the constant region may be used to modify disulfide linkages or oligosaccharide moieties that allow for enhanced localization due to increased antigen specificity or flexibility. The resulting physiological profile, bioavailability and other biochemical effects of the modifications, such as tumor localization, biodistribution and serum half-life, may easily be measured and quantified using well know immunological techniques without undue experimentation.

In certain embodiments, an Fc domain employed in an anti-PNAG antibody is an Fc variant. As used herein, the term “Fc variant” refers to an Fc domain having at least one amino acid substitution relative to the wild-type Fc domain from which said Fc domain is derived. For example, wherein the Fc domain is derived from a human IgG1 antibody, the Fc variant of said human IgG1 Fc domain comprises at least one amino acid substitution relative to said Fc domain.

The amino acid substitution(s) of an Fc variant may be located at any position (i.e., any EU convention amino acid position) within the Fc domain. In one embodiment, the Fc variant comprises a substitution at an amino acid position located in a hinge domain or portion thereof. In another embodiment, the Fc variant comprises a substitution at an amino acid position located in a CH2 domain or portion thereof. In another embodiment, the Fc variant comprises a substitution at an amino acid position located in a CH3 domain or portion thereof. In another embodiment, the Fc variant comprises a substitution at an amino acid position located in a CH4 domain or portion thereof.

The antibodies may employ any art-recognized Fc variant which is known to impart an improvement (e.g., reduction or enhancement) in effector function and/or FcR binding. Said Fc variants may include, for example, any one of the amino acid substitutions disclosed in International PCT Publications WO88/07089A1, WO96/14339A1, WO98/05787A1, WO98/23289A1, WO99/51642A1, WO99/58572A1, WO00/09560A2, WO00/32767A1, WO00/42072A2, WO02/44215A2, WO02/060919A2, WO03/074569A2, WO04/016750A2, WO04/029207A2, WO04/035752A2, WO04/063351A2, WO04/074455A2, WO04/099249A2, WO05/040217A2, WO05/070963A1, WO05/077981A2, WO05/092925A2, WO05/123780A2, WO06/019447A1, WO06/047350A2, and WO06/085967A2 or U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784, each of which is incorporated by reference herein. In one exemplary embodiment, an anti-PNAG antibody may comprise an Fc variant comprising an amino acid substitution at EU position 268 (e.g., H268D or H268E). In another exemplary embodiment, an anti-PNAG antibody may comprise an amino acid substitution at EU position 239 (e.g., S239D or S239E) and/or EU position 332 (e.g., I332D or I332Q).

In certain embodiments, an anti-PNAG antibody of may comprise an Fc variant comprising an amino acid substitution which alters the antigen-independent effector functions of the antibody, in particular the circulating half-life of the antibody. Such antibodies exhibit either increased or decreased binding to FcRn when compared to antibodies lacking these substitutions, therefore, have an increased or decreased half-life in serum, respectively. Fc variants with improved affinity for FcRn are anticipated to have longer serum half-lives, and such molecules have useful applications in methods of treating mammals where long half-life of the administered antibody is desired, e.g., to treat a chronic disease or disorder. In contrast, Fc variants with decreased FcRn binding affinity are expected to have shorter half-lives, and such molecules are also useful, for example, for administration to a mammal where a shortened circulation time may be advantageous, e.g. for in vivo diagnostic imaging or in situations where the starting antibody has toxic side effects when present in the circulation for prolonged periods. Fc variants with decreased FcRn binding affinity are also less likely to cross the placenta and, thus, are also useful in the treatment of diseases or disorders in pregnant women. In addition, other applications in which reduced FcRn binding affinity may be desired include those applications in which localization the brain, kidney, and/or liver is desired. In one exemplary embodiment, the altered antibodies exhibit reduced transport across the epithelium of kidney glomeruli from the vasculature. In another embodiment, the altered antibodies exhibit reduced transport across the blood brain barrier (BBB) from the brain, into the vascular space. In one embodiment, an antibody with altered FcRn binding comprises an Fc domain having one or more amino acid substitutions within the “FcRn binding loop” of an Fc domain. The FcRn binding loop is comprised of amino acid residues 280-299 (according to EU numbering). Exemplary amino acid substitutions which altered FcRn binding activity are disclosed in International PCT Publication No. WO05/047327 which is incorporated by reference herein. In certain exemplary embodiments, the antibodies, or fragments thereof, comprise an Fc domain having one or more of the following substitutions: V284E, H285E, N286D, K290E and S304D (EU numbering).

In other embodiments, antibodies, for use in the diagnostic and treatment methods described herein have a constant region, e.g., an IgG1 or IgG4 heavy chain constant region, which is altered to reduce or eliminate glycosylation. For example, an antibody may also comprise an Fc variant comprising an amino acid substitution which alters the glycosylation of the antibody. For example, said Fc variant may have reduced glycosylation (e.g., N- or O-linked glycosylation). In exemplary embodiments, the Fc variant comprises reduced glycosylation of the N-linked glycan normally found at amino acid position 297 (EU numbering). In another embodiment, the antibody has an amino acid substitution near or within a glycosylation motif, for example, an N-linked glycosylation motif that contains the amino acid sequence NXT or NXS. In a particular embodiment, the antibody comprises an Fc variant with an amino acid substitution at amino acid position 228 or 299 (EU numbering). In more particular embodiments, the antibody comprises an IgG1 or IgG4 constant region comprising an S228P and a T299A mutation (EU numbering).

Exemplary amino acid substitutions which confer reduce or altered glycosylation are disclosed in International PCT Publication No. WO05/018572, which is incorporated by reference herein. In preferred embodiments, the antibodies, or fragments thereof, are modified to eliminate glycosylation. Such antibodies, or fragments thereof, may be referred to as “agly” antibodies, or fragments thereof, (e.g. “agly” antibodies). While not being bound by theory, it is believed that “agly” antibodies, or fragments thereof, may have an improved safety and stability profile in vivo. Exemplary agly antibodies, or fragments thereof, comprise an aglycosylated Fc region of an IgG4 antibody which is devoid of Fc-effector function thereby eliminating the potential for Fc mediated toxicity to the normal vital organs. In yet other embodiments, antibodies, or fragments thereof, comprise an altered glycan. For example, the antibody may have a reduced number of fucose residues on an N-glycan at Asn297 of the Fc region, i.e., is afucosylated. In another embodiment, the antibody may have an altered number of sialic acid residues on the N-glycan at Asn297 of the Fc region.

iii) Covalent Attachment

Anti-PNAG antibodies may be modified, e.g., by the covalent attachment of a molecule to the antibody such that covalent attachment does not prevent the antibody from specifically binding to its cognate epitope. For example, but not by way of limitation, the antibodies, or fragments thereof, may be modified by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, etc. Additionally, the derivative may contain one or more non-classical amino acids.

Antibodies, or fragments thereof, may further be recombinantly fused to a heterologous polypeptide at the N- or C-terminus or chemically conjugated (including covalent and non-covalent conjugations) to polypeptides or other compositions. For example, anti-PNAG antibodies may be recombinantly fused or conjugated to molecules useful as labels in detection assays and effector molecules such as heterologous polypeptides, drugs, radionuclides, or toxins. See, e.g., PCT publications WO 92/08495; WO 91/14438; WO 89/12624; U.S. Pat. No. 5,314,995; and EP 396,387.

Anti-PNAG antibodies may be fused to heterologous polypeptides to increase the in vivo half life or for use in immunoassays using methods known in the art. For example, in one embodiment, PEG can be conjugated to the anti-PNAG antibodies to increase their half-life in vivo. Leong, S. R., et al., Cytokine 16:106 (2001); Adv. in Drug Deliv. Rev. 54:531 (2002); or Weir et al., Biochem. Soc. Transactions 30:512 (2002).

Moreover, anti-PNAG antibodies can be fused to marker sequences, such as a peptide to facilitate their purification or detection. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “flag” tag.

Anti-PNAG antibodies may be used in non-conjugated form or may be conjugated to at least one of a variety of molecules, e.g., to improve the therapeutic properties of the molecule, to facilitate target detection, or for imaging or therapy of the patient. Anti-PNAG antibodies can be labeled or conjugated either before or after purification, when purification is performed. In particular, anti-PNAG antibodies may be conjugated to therapeutic agents, prodrugs, peptides, proteins, enzymes, viruses, lipids, biological response modifiers, pharmaceutical agents, or PEG.

The present invention further encompasses anti-PNAG antibodies conjugated to a diagnostic or therapeutic agent. The anti-PNAG antibodies can be used diagnostically to, for example, monitor the development or progression of a immune cell disorder (e.g., CLL) as part of a clinical testing procedure to, e.g., determine the efficacy of a given treatment and/or prevention regimen. Detection can be facilitated by coupling the anti-PNAG antibodies to a detectable substance. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. See, for example, U.S. Pat. No. 4,741,900 for metal ions which can be conjugated to antibodies for use as diagnostics according to the present invention. Examples of suitable enzymes include horseradish peroxidase, alkaline phosphatase, .beta.-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include 125I, 131I, 111In or 99Tc.

Anti-PNAG antibodies for use in the diagnostic and treatment methods disclosed herein may be conjugated to cytotoxins (such as radioisotopes, cytotoxic drugs, or toxins) therapeutic agents, cytostatic agents, biological toxins, prodrugs, peptides, proteins, enzymes, viruses, lipids, biological response modifiers, pharmaceutical agents, immunologically active ligands (e.g., lymphokines or other antibodies wherein the resulting molecule binds to both the neoplastic cell and an effector cell such as a T cell), or PEG.

In another embodiment, an anti-PNAG antibody for use in the diagnostic and treatment methods disclosed herein can be conjugated to a molecule that decreases tumor cell growth. In other embodiments, the disclosed compositions may comprise antibodies, or fragments thereof, coupled to drugs or prodrugs. Still other embodiments of the present invention comprise the use of antibodies, or fragments thereof, conjugated to specific biotoxins or their cytotoxic fragments such as ricin, gelonin, Pseudomonas exotoxin or diphtheria toxin. The selection of which conjugated or unconjugated antibody to use will depend on the type and stage of cancer, use of adjunct treatment (e.g., chemotherapy or external radiation) and patient condition. It will be appreciated that one skilled in the art could readily make such a selection in view of the teachings herein.

It will be appreciated that, in previous studies, anti-tumor antibodies labeled with isotopes have been used successfully to destroy tumor cells in animal models, and in some cases in humans. Exemplary radioisotopes include: 90Y, 125I, 131I, 123I, 111In, 105Rh, 153Sm, 67Cu, 67Ga, 166Ho, 177Lu, 186Re and 188Re. The radionuclides act by producing ionizing radiation which causes multiple strand breaks in nuclear DNA, leading to cell death. The isotopes used to produce therapeutic conjugates typically produce high energy alpha- or beta-particles which have a short path length. Such radionuclides kill cells to which they are in close proximity, for example neoplastic cells to which the conjugate has attached or has entered. They have little or no effect on non-localized cells. Radionuclides are essentially non-immunogenic.

### VI. PHARMACEUTICAL FORMULATIONS AND METHODS OF ADMINISTRATION OF ANTI-PNAG ANTIBODIES

Methods of preparing and administering anti-PNAG antibodies, or fragments thereof, to a subject are well known to or are readily determined by those skilled in the art. The route of administration of the antibodies, or fragments thereof, may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope, a form for administration would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the polypeptides can be delivered directly to the site of the adverse cellular population thereby increasing the exposure of the diseased tissue to the therapeutic agent.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like. More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., an antibody by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or at risk of obtaining a PNAG-conatining microbial biofilm.

Effective doses of the stabilized antibodies, or fragments thereof, of the present invention, for the treatment of the above described conditions vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy.

For passive immunization with an antibody, the dosage may range, e.g., from about 0.0001 to 100 mg/kg, and more usually 0.01 to 5 mg/kg (e.g., 0.02 mg/kg, 0.25 mg/kg, 0.5 mg/kg, 0.75 mg/kg, 1 mg/kg, 2 mg/kg, etc.), of the host body weight. For example dosages can be 1 mg/kg body weight or 10 mg/kg body weight or within the range of 1-10 mg/kg, preferably at least 1 mg/kg. Doses intermediate in the above ranges are also intended to be within the scope of the invention.

Subjects can be administered such doses daily, on alternative days, weekly or according to any other schedule determined by empirical analysis. An exemplary treatment entails administration in multiple dosages over a prolonged period, for example, of at least six months. Additional exemplary treatment regimes entail administration once per every two weeks or once a month or once every 3 to 6 months. Exemplary dosage schedules include 1-10 mg/kg or 15 mg/kg on consecutive days, 30 mg/kg on alternate days or 60 mg/kg weekly. In some methods, two or more monoclonal antibodies with different binding specificities are administered simultaneously, in which case the dosage of each antibody administered may fall within the ranges indicated.

Anti-PNAG antibodies, or fragments thereof, can be administered on multiple occasions. Intervals between single dosages can be, e.g., daily, weekly, monthly or yearly. Intervals can also be irregular as indicated by measuring blood levels of polypeptide or target molecule in the patient. In some methods, dosage is adjusted to achieve a certain plasma antibody or toxin concentration, e.g., 1-1000 ug/ml or 25-300 ug/ml. Alternatively, antibodies, or fragments thereof, can be administered as a sustained release formulation, in which case less frequent administration is required. Dosage and frequency vary depending on the half-life of the antibody in the patient. In general, humanized antibodies show the longest half-life, followed by chimeric antibodies and nonhuman antibodies. In one embodiment, the antibodies, or fragments thereof, can be administered in unconjugated form. In another embodiment, the antibodies can be administered multiple times in conjugated form. In still another embodiment, the antibodies, or fragments thereof, can be administered in unconjugated form, then in conjugated form, or vise versa.

The dosage and frequency of administration can vary depending on whether the treatment is prophylactic or therapeutic. In prophylactic applications, compositions containing the present antibodies or a cocktail thereof are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time. Some patients continue to receive treatment for the rest of their lives.

In therapeutic applications, a relatively high dosage (e.g., from about 1 to 400 mg/kg of antibody per dose, with dosages of from 5 to 25 mg being more commonly used for radioimmunoconjugates and higher doses for cytotoxin-drug conjugated molecules) at relatively short intervals is sometimes required until progression of the disease is reduced or terminated, and preferably until the patient shows partial or complete amelioration of symptoms of disease. Thereafter, the patent can be administered a prophylactic regime.

In one embodiment, a subject can be treated with a nucleic acid molecule encoding a an anti-PNAG antibody (e.g., in a vector). Doses for nucleic acids encoding polypeptides range from about 10 ng to 1 g, 100 ng to 100 mg, 1 ug to 10 mg, or 30-300 ug DNA per patient. Doses for infectious viral vectors vary from 10-100, or more, virions per dose.

Therapeutic agents can be administered by parenteral, topical, intravenous, oral, subcutaneous, intraarterial, intracranial, intraperitoneal, intranasal or intramuscular means for prophylactic and/or therapeutic treatment. Intramuscular injection or intravenous infusion are preferred for administration of an antibody. In some methods, therapeutic antibodies, or fragments thereof, are injected directly into the cranium. In some methods, antibodies, or fragments thereof, are administered as a sustained release composition or device, such as a Medipad™ device.

Anti-PNAG antibodies can optionally be administered in combination with other agents that are effective in treating the disorder or condition in need of treatment (e.g., prophylactic or therapeutic). Preferred additional agents are those which are art recognized and are standardly administered for a particular disorder.

Effective single treatment dosages (i.e., therapeutically effective amounts) of 90Y-labeled antibodies range from between about 5 and about 75 mCi, more preferably between about 10 and about 40 mCi. Effective single treatment non-marrow ablative dosages of 131I-labeled antibodies range from between about 5 and about 70 mCi, more preferably between about 5 and about 40 mCi. Effective single treatment ablative dosages (i.e., may require autologous bone marrow transplantation) of 131I-labeled antibodies range from between about 30 and about 600 mCi, more preferably between about 50 and less than about 500 mCi. In conjunction with a chimeric modified antibody, owing to the longer circulating half life vis-a-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the 111In label, are typically less than about 5 mCi.

While a great deal of clinical experience has been gained with 131I and 0.90Y, other radiolabels are known in the art and have been used for similar purposes. Still other radioisotopes are used for imaging. For example, additional radioisotopes which are compatible with the scope of the instant invention include, but are not limited to, 123I, 125I, 32P, 57Co, 64Cu, 67Cu, 77Br, 81Rb, 81Kr, 87Sr, 113In, 127Cs, 129Cs, 1321, 197Hg, 203Pb, 206Bi, 177Lu, 186Re, 212Pb, 212Bi, 47Sc, 105Rh, 109Pd, 153Sm, 188Re, 199Au, 225Ac, 211A 213Bi. In this respect alpha, gamma and beta emitters are all compatible with in the instant invention. Further, in view of the instant disclosure it is submitted that one skilled in the art could readily determine which radionuclides are compatible with a selected course of treatment without undue experimentation. To this end, additional radionuclides which have already been used in clinical diagnosis include 125I, 123I, 99Tc, 43K, 52Fe, 67Ga, 68Ga, as well as 111In. Antibodies have also been labeled with a variety of radionuclides for potential use in targeted immunotherapy (Peirersz et al. Immunol. Cell Biol. 65: 111-125 (1987)). These radionuclides include 188Re and 186Re as well as 199Au and 67Cu to a lesser extent. U.S. Pat. No. 5,460,785 provides additional data regarding such radioisotopes and is incorporated herein by reference.

As previously discussed, the antibodies, or fragments thereof, can be administered in a pharmaceutically effective amount for the in vivo treatment of mammalian disorders. In this regard, it will be appreciated that the disclosed antibodies, or fragments thereof, will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of the instant application, a pharmaceutically effective amount of an antibody, conjugated or unconjugated to a therapeutic agent, shall be held to mean an amount sufficient to achieve effective binding to a target and to achieve a benefit, e.g., to ameliorate symptoms of a disease or disorder or to detect a substance or a cell. In the case of tumor cells, the polypeptide will be preferably be capable of interacting with selected immunoreactive antigens on neoplastic or immunoreactive cells and provide for an increase in the death of those cells. Of course, the pharmaceutical compositions of the present invention may be administered in single or multiple doses to provide for a pharmaceutically effective amount of the polypeptide.

In keeping with the scope of the present disclosure, anti-PNAG antibodies may be administered to a human or other animal in accordance with the aforementioned methods of treatment in an amount sufficient to produce a therapeutic or prophylactic effect. The anti-PNAG antibodies can be administered to such human or other animal in a conventional dosage form prepared by combining the antibody with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of polypeptides according to the present invention may prove to be particularly effective.

### VII. METHODS OF TREATING OR PREVENTING INFECTIONS INVOLVING PNAG-CONTAINING MICROBIAL BIOFILM

The invention provides methods for treating or preventing PNAG-containing bacterial biofilm by administering to a subject in need of thereof a pharmaceutical composition comprising one or more anti-PNAG antibody, or antigen binding fragment thereof.

In certain embodiments, a pharmaceutical composition comprising one or more anti-PNAG antibodies, or antigen binding fragments thereof, are administered to a subject in combination with one or more additional therapeutic agents. In a particular embodiments, the one or more additional therapeutic agents is administered concurrently with the pharmaceutical composition comprising one or more anti-PNAG antibodies. Suitable additional therapeutic agents include antibacterial agents (e.g., antibiotics). Suitable anti-bacterial agents include, without limitation, penicillin G, penicillin V, ampicillin, amoxicillin, bacampicillin, cyclacillin, epicillin, hetacillin, pivampicillin, methicillin, nafcillin, oxacillin, cloxacillin, dicloxacillin, flucloxacillin, carbenicillin, ticarcillin, avlocillin, mezlocillin, piperacillin, amdinocillin, cephalexin, cephradine, cefadoxil, cefaclor, cefazolin, cefuroxime axetil, cefamandole, cefonicid, cefoxitin, cefotaxime, ceftizoxime, cefinenoxine, ceftriaxone, moxalactam, cefotetan, cefoperazone, ceftazidme, imipenem, clavulanate, timentin, sulbactam, neomycin, erythromycin, metronidazole, chloramphenicol, clindamycin, lincomycin, vancomycin, trimethoprim-sulfamethoxazole, aminoglycosides, quinolones, tetracyclines and rifampin. (See e.g., Goodman and Gilman's, Pharmacological Basics of Therapeutics, 8th Ed., 1993, McGraw Hill Inc.)

One skilled in the art would be able, by routine experimentation, to determine what an effective, non-toxic amount of antibody (or additional therapeutic agent) would be for the purpose of treating a PNAG-associated disease or disorder. For example, a therapeutically active amount of a polypeptide may vary according to factors such as age, sex, medical complications (e.g., immunosuppressed conditions or diseases) and weight of the subject, and the ability of the antibody to elicit a desired response in the subject. The dosage regimen may be adjusted to provide the optimum therapeutic response. For example, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

The precise time of administration of the anti-PNAG antibody can be adjusted according to the patients needs. In the case of prophylactic treatment to inhibit the formation of a PNAG-containing microbial biofilm in a subject, the anti-PNAG antibody can be given at any time prior to exposure to the biofilm-forming microbe (e.g, upon entry into hospital or at the start of a medical procedure). For example, the anti-PNAG antibody can be administered between 0 and 240 hours prior to exposure to the biofilm-forming microbe, e.g., about 0.5, 1, 2, 3, 4, 5, 6, 7, 8, 9, 10, 11, 12, 13, 14, 15, 16, 17, 18, 19, 20, 21, 22, 23 24, 30, 40, 60, 80, 90, 100, 110, 110, 120, 130, 140, 150, 160, 170, 180, 190, 200, 2010, 220, 230, and/or 240 hours.

### VIII. EXEMPLIFICATION

The present invention is further illustrated by the following examples which should not be construed as further limiting. The contents of Sequence Listing, figures and all references, patents and published patent applications cited throughout this application are expressly incorporated herein by reference.

### Example 1

### Inhibition of S. aureus Biofilm by F598

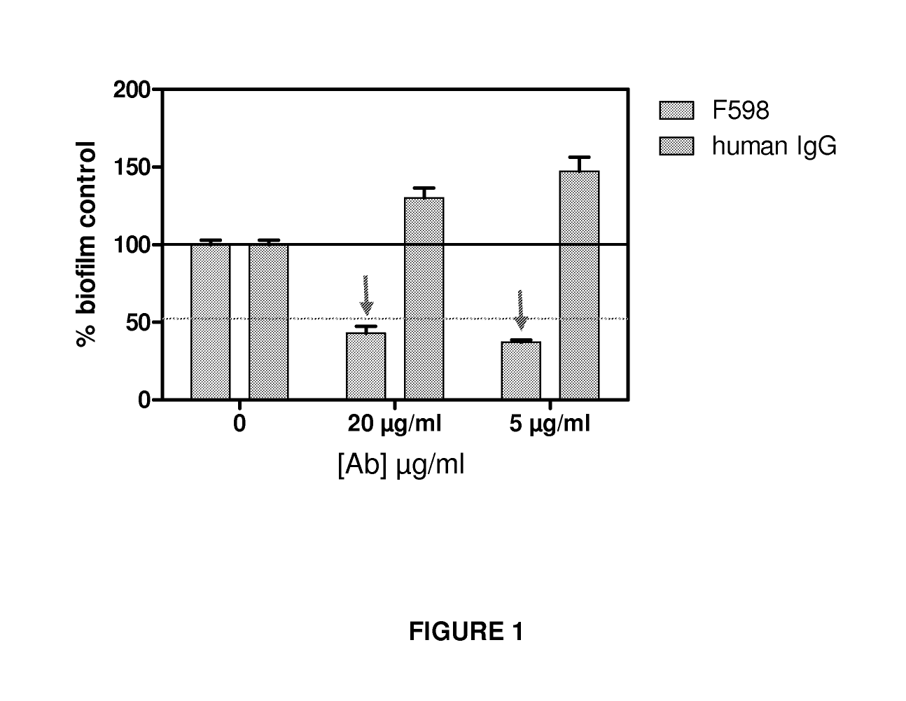

S. aureus (ATCC 33592) was grown overnight in TSB medium, adjusted at 1E+07 CFU/ml. The bacteria were then aliquoted into MBEC plates (MBEC Assays™, Innovotech) containing 5, or 20 ug/ml of concentration of F598 or human IgG control and incubated for 5.5 h. Following the incubation period, the pegs of the MBEC plates were removed, washed and the amount of formed biofilm quantified using by luminescence (BacTiter-Glo™ Microbial Cell Viability Assay, Promega). In this experiment F598 demonstrated a clear protective effect against S. aureus in vitro biofilm development. Specifically, F598 showed more than 50% inhibition of S. aureus biofilm development (43+/−8% and 37+/−3%, at 5 and 20 μg/ml, respectively) (see FIG. 1).

### Example 2

### Inhibition of S. epidermidis (Clinical Isolate 1457) Biofilm by F598

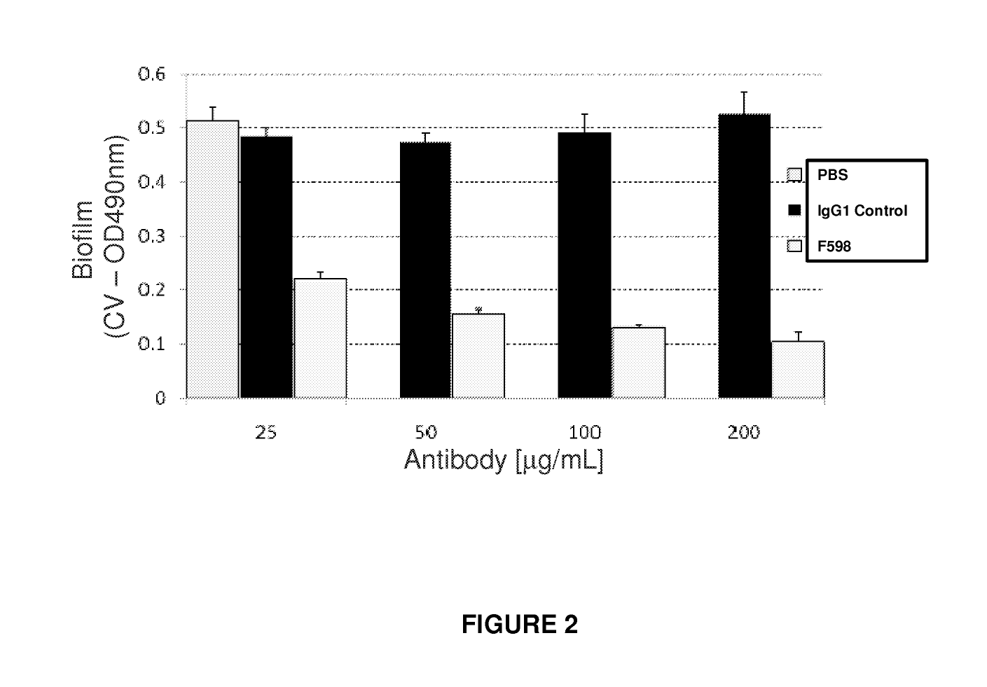

S. epidermidis (clinical isolate 1457) was grown in multiwell plates in TSB medium for 8 h in the presence of 25, 50, 100, or 200 μg/ml of F598 or human IgG1 control. Wells were then washed and stained with crystal violet. Biofilms were quantified by measuring the sample absorbance at a wavelength of 490 nm. In this experiment F598 demonstrated a clear, dose-dependent protective effect against S. epidermidis 1457 in vitro biofilm development. Specifically, at 25 μg/ml biofilm formation was 40% of the control (0.2 vs 0.50D), and at 200 μg/ml biofilm formation was 20% of the control (0.1 vs 0.50D) (see FIG. 2).

### Example 3

### Inhibition of S. epidermidis (Clinical Isolate RP62A) Biofilm by F598

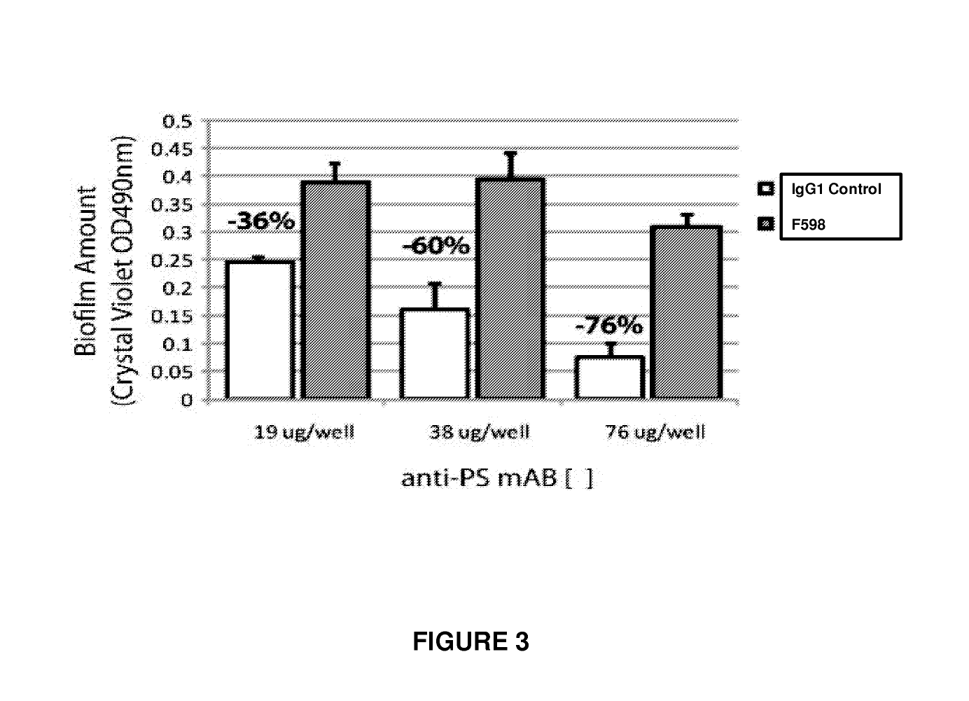

S. epidermidis (clinical isolate RP62A) was grown in multiwell plates in TSB medium for 8 h in the presence of 19, 38, or 76 μg/ml of F598 or PBS buffer control. Wells were then washed and stained with crystal violet. Biofilms were quantified by measuring the sample absorbance at a wavelength of 490 nm. In this experiment F598 demonstrated a clear, dose-dependent protective effect against S. epidermidis RP62A in vitro biofilm development. Specifically, 19, 38, and 76 μg/ml of F598 reduced biofilm formation 36, 60 and 76%, respectively (relative to the corresponding PBS control) (see FIG. 3).

We claim:

1. A method of preventing a nocosomial infection comprising:
identifying a subject at risk of developing an poly-N-acetyl glucosamine (PNAG)-containing microbial biofilm from a medical procedure; and
administering to the subject an effective amount of an antibody that specifically binds to PNAG and inhibits PNAG-containing microbial biofilm formation, thereby preventing the nosocomial infection.

2. The method of claim 1, wherein the nocosomial infection is a lung infection, joint infection, endocardial infection, skin infection, soft tissue infection, or septicemia.

3. The method of claim 1 or 2, wherein the antibody is administered between about 0 and 240 hours prior to the medical procedure.

4. The method of any one of claims 1-3, wherein the medical procedure is the installation of a surgical implant in the subject.

5. The method of claim 4, wherein the surgical implant is a stent, catheter, cannula, prosthesis, or pace-maker.

6. The method of claim 4 or 5, wherein the antibody is administered between about 0 and 240 hours prior to implantation of surgical implant.

7. The method of any one of the preceding claims, wherein the antibody is administered systemically.

8. The method of any one of claims 1-6, wherein the antibody is administered locally to the site of implantation of the surgical implant.

9. The method of any one of claims 1-6, wherein the antibody is coated on the surgical implant or adhered to the surgical implant.

10. The method of any one of the preceding claims, wherein the antibody is administered in a controlled release formulation.

11. The method of any one of the preceding claims, wherein the subject is a human.

12. The method of any one of the preceding claims, wherein the biofilm comprises Staphylococcus.

13. The method of claim 12, wherein the Staphylococcus is S. epidermidis or S. aureus.

14. The method of any one of the preceding claims, wherein the antibody is a human antibody.

15. The method of any one of the preceding claims, wherein the antibody comprises a VH domain comprising the HCDR1, HCDR2, and HCDR3 amino acid sequences set forth in SEQ ID Nos: 1, 2, and 3, respectively.

16. The method of any one of the preceding claims, wherein the antibody comprises a VL domain comprising the LCDR1, LCDR2, and LCDR3 amino acid sequences set forth in SEQ ID Nos: 4, 5, and 6, respectively.

17. The method of any one of the preceding claims, wherein the antibody comprises the VH domain sequence set forth in SEQ ID No: 7.

18. The method of any one of the preceding claims, wherein the antibody comprises the VL domain sequence set forth in SEQ ID No: 8.

19. A method of inhibiting formation of a poly N-acetyl glucosamine (PNAG)-containing microbial biofilm on a surgical implant in a subject, the method comprising administering to the subject an effective amount of an antibody that specifically binds to PNAG prior to implantation of the surgical implant into the subject.

20. The method of claim 19, wherein the surgical implant is a stent, catheter, cannula, prosthesis, or pace-maker.

21. The method of claim 19 or 20, wherein the antibody is administered between about 0 and 240 hours prior to the medical procedure.

22. The method of any one of claims 19-21, wherein the antibody is administered systemically.

23. The method of any one claims 19-21, wherein the antibody is administered locally to the site of implantation of the surgical implant.

24. The method of any one claims 19-21, wherein the antibody is coated on the surgical implant or adhered to the surgical implant.

25. The method of any one claims 19-24, wherein the antibody is administered in a controlled release formulation.

26. The method of any one of claims 19-25, wherein the subject is a human.

27. The method of any one of claims 19-26, wherein the biofilm comprises Staphylococcus.

28. The method of claim 27, wherein the Staphylococcus is S. epidermidis or S. aureus.

29. The method of any one of claims 19-28, wherein the antibody is a human antibody.

30. The method of any one of claims 19-29, wherein the antibody comprises a VH domain comprising the HCDR1, HCDR2, and HCDR3 amino acid sequences set forth in SEQ ID Nos: 1, 2, and 3, respectively.

31. The method of any one of claims 19-30, wherein the antibody comprises a VL domain comprising the LCDR1, LCDR2, and LCDR3 amino acid sequences set forth in SEQ ID Nos: 4, 5, and 6, respectively.

32. The method of any one of claims 19-31, wherein the antibody comprises the VH domain sequence set forth in SEQ ID No: 7.

33. The method of any one of claims 19-32, wherein the antibody comprises the VL domain sequence set forth in SEQ ID No: 8.

34. A method of inhibiting formation of PNAG-containing microbial biofilm on a substrate comprising contacting the substrate with an effective amount of an antibody that specifically binds to PNAG.

35. The method of claim 34, wherein the biofilm comprises Staphylococcus.

36. The method of claim 35, wherein the Staphylococcus is S. epidermidis or S. aureus.

37. The method of any one of claims 34-36, wherein the antibody is a human antibody.

38. The method of any one of claims 34-37, wherein the antibody comprises a VH domain comprising the HCDR1, HCDR2, and HCDR3 amino acid sequences set forth in SEQ ID Nos: 1, 2, and 3, respectively.

39. The method of any one of claims 34-38, wherein the antibody comprises a VL domain comprising the LCDR1, LCDR2, and LCDR3 amino acid sequences set forth in SEQ ID Nos: 4, 5, and 6, respectively.

40. The method of any one of claims 34-39, wherein the antibody comprises the VH domain sequence set forth in SEQ ID No: 7.

41. The method of any one of claims 34-40, wherein the antibody comprises the VL domain sequence set forth in SEQ ID No: 8.
